(19) United States
(12) Patent Application Publication
(10) Pub. No.: US 2002/0168657 A1
(43) Pub. Date: Nov. 14, 2002

(54) Rare event detection system

(76) Inventors: Lan Bo Chen, Lexington, MA (US); Stine-Kathrein Kraeft, Boston, MA (US); Daniel Auclair, Ashland, MA (US)

Correspondence Address: JEFFREY D. HSI, Fish & Richardson P.C., 225 Franklin Street, Boston, MA, 02110-2804, US

(21) Appl. No.: 10/062,197

(22) Filed: Feb. 1, 2002

Related U.S. Application Data
(60) Provisional application No. 60/265,909, filed on Feb. 2, 2001.

(51) Int. Cl. C12Q 1/68; G06F 19/00; G01N 33/48; G01N 33/50; C12M 1/34

(52) U.S. Cl. 435/6; 435/287.2; 702/20

(57) ABSTRACT

Disclosed is a method of detecting a target body in a specimen field of multiple candidate bodies by differential fluorescence labeling of different portions of the target body. The flexibility provided by the use of fluorophores allows for rapid detection of any rare target body with high efficiency and accuracy.

### CROSS REFERENCE TO RELATED APPLICATIONS

This application claims benefit of U.S. application Ser. No. 60/265,909, entitled Rare Event Detection System, filed Feb. 2, 2001, which application is hereby incorporated by reference in its entirety.

### STATEMENT AS TO FEDERALLY SPONSORED RESEARCH

This invention was made with Government funds through a grant (CA13849) from the National Institutes of Health. The Government has certain rights in the invention.

### BACKGROUND OF THE INVENTION

Most human cancers are characterized by the aberrant expression of normal and/or mutated genes, and natural selection acts on cancer cells to cause a loss of growth control, angiogenesis, invasion, and metastasis. Thus, the ability to detect cancer cells of particular phenotypes in patient samples provides valuable information to a health care provider. For example, if the presence of metastatic cancer cells is detected in the body, then a medical professional might consider a more aggressive therapy for the patient.

Cancer cell detection methods that rely on expression of cancer markers generally require long, labor-intensive, and sometimes expensive immunohistochemistry or nucleic acid hybridization procedures that, though ubiquitous in research laboratories, are less accessible in the clinic. Furthermore, in many instances the particular marker being screened is only produced, either initially, or in detectable levels only at a late stage of cancer progression, such that the advantage of early detection is squandered. Current technologies allow detection of micrometastasis along the order of 1 parts-per-million (i.e., one cancer cell per one million other cells), however, this detection level is still inadequate for true “early detection” in certain cancers. More sensitive levels of detection would effectively provide cancer cell detection capabilities to allow appropriate and more effective intervention of cancer cell proliferation and thereby more effective and timely cancer treatment and disease modulation therapies. Thus, there is a need for fast, efficient, reliable, and sensitive detection methods that are more amenable for use in the clinic.

The detection of biological weapons (BW) on a battle field poses a similar problem, i.e., no suitable method or device for detecting a rare particle (e.g., toxin or virus) among a large population of particles. Biological weapons, defined as infectious agents such as bacteria and viruses or related toxins, when used intentionally to inflict harm upon others, have been with us for a long time. They were probably originally used in prehistoric times, as arrowheads dipped into plant or animal extracts containing toxins; or in fecal matter or decaying meat, which are sources of the gas gangrene bacterium, Clostridium perfringens, and often also of the tetanus bacillus, C. tetani. BW first appear on the record as early as the 6th Century BC when the Assyrians poisoned enemy wells with rye ergot; and Solon of Athens used the purgative herb hellebore (skunk cabbage) to poison the water supply during the siege of Krissa; the Romans and many others have used a similar strategy; and during the 14th Century AD, the Mongols are said to have catapulted plague-infected corpses over the city walls of Kaffa, which they were besieging, an event that may have started the Black Death pandemic that spread throughout Europe. Other examples for the crude or more sophisticated use of BW abound, up to the late 20th Century.

Advances in basic and applied microbiology now allow skilled scientists to harness and weaponize the most virulent pathogens and toxins. While several countries (including the United States) have developed BW programs at some point or another during the 20th century, efforts in Japan and in the former Soviet Union are perhaps the most notorious. From 1932 until the end of WW II, the Japanese Army engaged in biological weapons research through its “Unit 731,” based in occupied China. Research with human subjects (Chinese and Russian civilians and American, British, Chinese, Korean and Russian prisoners of war) was conducted using a variety of agents including anthrax, glanders, plague, typhoid, paratyphoid A and B, typhus, smallpox, tularemia, infectious jaundice, gas gangrene, tetanus, cholera, dysentery, scarlet fever, undulant fever, tick encephalitis, whooping cough, diphtheria, pneumonia, venereal diseases, tuberculosis and Salmonella. The Soviet program was initiated in 1928, when the governing Revolutionary Military Council signed a secret decree ordering the transformation of typhus into a battlefield weapon. In the 1930's, scientists at the Solovetsky Island facility, in the Arctic, worked with typhus, Q-fever, glanders, and melioidosis. From 1973 through at least the early 1990's, the Soviet Union carried out a program aimed at modernizing existing biological weapons and at developing genetically altered pathogens, resistant to antibiotics and vaccines, which could be turned into powerful weapons for use in intercontinental warfare. Agents studied included anthrax, turalemia, plague, glanders, smallpox, Ebola, Marburg, Machupo, Junin, and Venezuelan equine encephalitis.

All open societies, such as ours, are by their very nature vulnerable to terrorist attacks, both from international and domestic groups. With this state of affairs, it is most urgent that effective countermeasures be developed to preempt biological attacks, or render them ineffective through the protection of the target population (troops or civilians).

Biological weapons have a few unique features that make them especially formidable. For one, hurdles would be few for a small team comprising a competent microbiologist and a mechanical engineer, to grow or extract a variety of pathogenic agents (bacteria, viruses or toxins) and build an effective dispersion system: it has been estimated that a major biological arsenal could be built in a room 15 by 15 ft., with $10,000 worth of equipment. This makes BW tools of choice for groups bent on terrorism who may want to inflict massive casualties to their opponent. Also, contagion may in some cases expand the outcome of the attack well beyond the confines of the original hit, both geographically and temporally. Finally, the actions of BW agents on the victims are generally delayed by at least hours, usually days, allowing a covert attack to be sustained during this period (besides giving the perpetrators an opportunity to flee, another boon for the stealth terrorist), and the early symptoms of an infection with a variety of BW pathogens are flu-like, making it very difficult to quickly recognize a BW attack as such.

Our ability to respond effectively to a biological attack on an unimmunized population therefore depends crucially on the development of new modalities for the rapid monitoring of BW agents in the environment, both airborne (indoors and outdoors) and waterborne, before an outbreak of the disease. This is also the time window when early detection of pathogens in human body fluids, e.g., blood, prior to the appearance of clinical symptoms is important.

Theoretically, any pathogen could be used as a biological weapon. However, certain characteristics make a biological organism or a biologically derived bioactive substance (BDBS), such as bacterial toxins, especially suited for use as weapons of mass destruction. These agents can be: 1) highly infectious, contagious, and toxic (i.e., even low-level exposure causes disease); 2) efficiently dispersible, e.g., in the air; 3) readily grown and produced in large quantities; 4) stable in storage; 5) resistant to environmental conditions, for extended effect; and 6) resistant to treatment, e.g., antibiotics, antibodies, other drugs.

To the list of natural pathogens, one should add genetically modified BW agents. This class of agents is particularly dreadful because they would be generated to make them more potent, even creating new diseases (e.g., resulting in a “brainpox” virus), or produce pathogens resistant to existing countermeasures. These pose a special challenge due to their unpredictability.

For the reasons described above, a covert attack using BW agents would be extremely difficult to detect and assess (in the absence of intelligence). At the present time, as no formal environmental monitoring system exists, the earliest knowledge that an attack took place would occur in many cases only when victims start pouring into emergency rooms and an outbreak is recognized. This, of course, is far too late. The classical monitoring methods for pathogens involve environmental sampling (air, water supply) or body fluid sampling (blood, urine, sputum etc.) onto growth media and culture of the sample followed by a battery of microbiological tests to identify the culprit. In addition to the fact that culture is not a trivial endeavor (e.g., for viruses), such a procedure is much too lengthy to provide a timely alert. Other possible analysis methods include biochemical assays, immunoassays, “GeneChip” screening, and the polymerase chain reaction (PCR), but all these require amounts of the contaminant that may not be present in the initial sample (to meet a sensitivity commensurate with an actual threat), such that culture may still be needed; even PCR from a single bacterium or virion is impractical.

### SUMMARY OF THE INVENTION

The invention is based on technologies that provide for detecting the presence of a rare event or marker. The invention relates to equipment and methods for identifying, characterizing (either quantitatively, qualitatively, or both), analyzing or determining the presence of minute quantities of rare events or markers. The determination of the presence or absence of such rare events or markers, as well as the quantification of such rare events or markers, is useful in providing early detection of deleterious or potentially harmful entities or conditions, which if identified earlier rather than later, can allow for the application of an appropriate response, treatment, or other intervention regimen or protocol. Rare events include both normal events (e.g., the presence or absence of target bodies or cells that are present in normal physiological states) and abnormal events (e.g., the presence or absence of target bodies or cells that are present in abnormal physiological states such as those associated with disease, disease symptoms, or genetic abnormalities). One problem with current diagnostic methods, particularly for cancer, relates to minimal residual disease. That is, instances when the level of disease cells or other disease markers (e.g., nucleic acids, proteins, cell surface receptors) is too low for current detection methods, however, significant enough that they represent the potential for further proliferation, up-regulation or recurrence of the disease if left undiagnosed or untreated. Thus, in many instances, identification of disease risk (i.e., cancer, artherosclerosis, central nervous system disease, etc.) in a more timely manner would allow for earlier treatment, which leads to more effective treatments; or earlier identification of risk to populations (i.e., biological warfare agents), which allows for minimization of exposure and uncontrolled spreading or distribution of that risk to greater populations, is desirable.

The invention is based on the discovery of a highly sensitive and efficient method of detecting rare cancer cells in a large cell population. In addition, the cancer cell detection system implemented herein led to the realization that almost any rare target body within a large population of candidate bodies can be detected via this system, modified for the particular target body to be identified. The methods and systems of the invention rely on fluorescent labels that specifically bind to subsets of a large population, each subset including the target body to be detected. A target body is any body (e.g., a cell, a pathogen, a virus, a toxin, a prion) in the specimen field that is sought to be identified (e.g., by labeling, including directly to the target body or indirectly such as when the label is coupled to an molecule that binds or interacts with the target body). A candidate body is any body (e.g., a cell, a pathogen, a virus, a toxin, a prion) in the specimen field that is being analyzed.

Accordingly, the invention features a method of detecting a target body (e.g., a cancer cell) in a specimen by obtaining a specimen field (e.g., peripheral blood mononuclear cells (PBMC) or bone marrow cells spread out on a glass surface) exposed to or labeled with at least a first fluorophore and a second fluorophore, the first fluorophore emitting photons at a first wavelength and the second fluorophore emitting photons at a second wavelength; exposing the specimen field to light sufficient to excite the first and second fluorophores; scanning the specimen field for first sources of photons at the first wavelength and for second sources of photons at the second wavelength; acquiring and recording a first image of the specimen field at each location, the first image generated via an optical or electronic filter that substantially blocks photons of the second wavelength but is permissive for photons of the first wavelength and; indexing the corresponding location within the specimen field; acquiring and recording a second image of the specimen field at each location, the second image generated via an optical or electronic filter that substantially blocks photons of the first wavelength but is permissive for photons of the second wavelength; indexing the corresponding location within the specimen field; and retrieving and inspecting a first image and second image at a single location within the specimen field. The presence of a candidate body in the first and second images at the single location indicates the presence of a target body in the specimen. Images of different fluorescent signals can be overlaid for positive confirmation of the event or for phenotypic evaluation. The two scans can be run independently.

The first fluorophore can be a compound that specifically binds to DNA, such as DAPI, or RNA, such as acridine orange. The second fluorophore can be coupled to a molecule (e.g., an antibody or nucleic acid) that specifically binds to a cancer cell marker, such as cytokeratin or another marker.

In some embodiments, the specimen field can be labeled with a third fluorophore to increase the specificity of the rare event detection or to detect multiple subsets of target bodies, for example a cancer cell and a virus, and the method can further include exposing the specimen field to light sufficient to excite the third fluorophore, the third fluorophore emitting light at a third wavelength; scanning the specimen field for third sources of photons at the third wavelength; registering the location of each third source within the specimen field; acquiring and recording a third image of the specimen field at each location, the third image generated via an optical or electronic filter that substantially blocks photons of the first and second wavelength but is permissive for photons of the third wavelength; indexing each third image to the corresponding location within the specimen field; and retrieving and inspecting a third image at the single location within the specimen field. The presence of a candidate body in the first, second, and third images at the single location indicates the presence of a target body. The third fluorophore can be coupled to a molecule (e.g., an antibody) that specifically binds to a second cancer cell marker such as an epithelial cell adhesion molecule (e.g., Ep-CAM) or a disialo-ganglioside antigen (e.g., GD2).

The methods can further include counting the total number of locations in the specimen field that produced a first image, counting the total number of locations in the specimen field that produced both a first image and a second image, or counting the total number of locations in the specimen field that produced a first, second, and third image. In addition, the methods can include inspecting a first image and second image at another single location within the specimen field, where the presence of a candidate body in the first image and in the second image at the other single location indicates the present of a different target body.

The invention further features a detection system including a stage for receiving a specimen field; a detector (e.g., microscope) positioned and configured to acquire images of locations within the specimen field; a light source positioned and configured to expose the specimen field to light sufficient to excite a first fluorophore at a first excitation wavelength and sufficient to excite a second fluorophore at a second excitation wavelength; a camera attached to the detector (e.g., microscope), the camera positioned and configured to (1) capture a first image at a location in the specimen field via an optical or electronic filter that substantially blocks photons at a second emission wavelength of the second fluorophore but is permissive for photons at a first emission wavelength of the first fluorophore, and (2) capture a second image at the location in the specimen field via an optical or electronic filter that substantially blocks photons at the first emission wavelength but is permissive for photons at the second emission wavelength; and a computer that records the first image and second image and indexes the first image and second image to the corresponding location within the specimen field, the computer displaying, on demand by a user, the first image and second image for the corresponding location.

The stage can be movable about three perpendicular axes and addressable in at least two of the three axes. Alternatively, the camera or a housing containing the camera and/or image capture device can be movable about three perpendicular axes and addressable in at least two of the three axes. The camera can include a charge-coupled device for capturing the first and second images or a plurality of optical filters for use in capturing the first and second images. Alternatively or in conjunction with optical filters, the cameral or computer can include electronic filters. Such filters can dissect a digitized color image taken at a range of wavelengths (e.g., the visible wavelengths) into images formed at only specific wavelengths or narrower ranges of wavelengths.

In another aspect, the invention features a method of detecting a target body in a specimen by obtaining a specimen field labeled with at least a first fluorophore, the first fluorophore emitting photons at a first wavelength; exposing the specimen field to light sufficient to excite the first fluorophore; scanning the specimen field at a low magnification for first sources of photons at the first wavelength; acquiring and recording a first image of the specimen field at each location; indexing each first image to the corresponding location within the specimen field; and inspecting a first image at a single location within the specimen field, where the presence of a candidate body in the first image at the single location indicates the presence of a target body in the specimen.

The methods and systems of the invention are capable of fast, highly sensitive, and efficient detection of rare target bodies within a large population of candidate bodies, such as a rare cancer cell within a million healthy cells, a level of sensitivity achievable with the present invention. The methods and systems herein allow for detection levels along the order of about 0.1 parts-per-million, or commensurately more beneficial, about 0.05, about 0.03, or about 0.01 parts-per-million.

In one aspect the invention is a method of detecting the presence or absence of a target body in a specimen, the method comprising

obtaining a specimen field exposed to or labeled with at least a first fluorophore and a second fluorophore, the first fluorophore emitting photons at a first wavelength and the second fluorophore emitting photons at a second wavelength;

exposing the specimen field to light sufficient to excite the first and second fluorophores;

scanning the specimen field at a low magnification for first sources of photons at the first wavelength and for second sources of photons at the second wavelength;

registering the location of each first source and each second source within the specimen field;

acquiring and recording a first image of the specimen field at each location, the first image generated via an optical or electronic filter that substantially blocks photons of the second wavelength but is permissive for photons of the first wavelength;

acquiring and recording a second image of the specimen field at each location at a high magnification, the second image generated via an optical or electronic filter that substantially blocks photons of the first wavelength but is permissive for photons of the second wavelength;

indexing each first image and each second image to the corresponding location within the specimen field; and

inspecting a first image and second image at a single location within the specimen field,

wherein the presence of a candidate body in the first and second images at the single location indicates the presence of a target body in the specimen.

In another aspect the invention is any method herein wherein preparation of the specimen field comprises:

a. lysing the cell sample to give a sample mixture;

b. centrifuging the sample mixture;

c. separating the supernatant from the sample mixture;

d. resuspending the resulting pellet of cells in a physiological buffer solution;

e. plating the cells on an adhesive slide;

f. adding cell culture media to the slide.

and wherein preparation of the specimen field further comprises:

after step d, making a dilution of the cell mixture, treating the dilution with a dye sensitive for dead cells, performing a cell count to determine the sample cell density for the slide to be used.

In other aspects, the methods are any of those herein: wherein the target body is a cancer, epithelial, smooth muscle, dendritic, memory T-, memory B-, somatic, normal, aberrant, or stem cell; wherein the system is capable of detecting at least one target cell in a specimen field of at least 1,000,000 cells; wherein the system is capable of detecting at least one target cell in a specimen field of at least 25,000,000 cells; wherein the system is capable of detecting at least one target cell in a specimen field of at least 50,000,000 cells; wherein the system is capable of detecting at least one target cell in a specimen field of at least 100,000,000 cells; wherein the recording comprises at least a 1024×1024 pixel array image; or wherein the recording comprises at least a 1600×1600 pixel array image.

In other aspects, the methods are any of those herein: wherein the field specimen comprises white blood cells as the majority of cell types; wherein the field specimen comprises heterogeneous cells types; wherein the field specimen comprises macrophages; wherein the specimen field is an environmental sample; wherein the light is ultraviolet light, infrared light, or visible light; wherein the target body is a cancer cell, and the specimen field is white blood cells or bone marrow cells spread out on a glass surface; wherein the first fluorophore is a compound that specifically binds to DNA; wherein the second fluorophore is coupled to a molecule that specifically binds to a cancer cell marker; wherein the cancer cell marker is cytokeratin; wherein the cancer cell marker resides in the cytoplasm; wherein the cancer cell surface marker is an epithelial cell adhesion molecule; wherein the cancer cell surface marker is a disialo-ganglioside antigen; further comprising counting the total number of locations in the specimen field that produced a first image; further comprising counting the total number of locations in the specimen field that produced both a first image and a second image; further comprising counting the total number of locations in the specimen field that produced a first, second, and third image; further comprising inspecting a first image and second image at another single location within the specimen field, wherein the presence of a candidate body in the first image and in the second image at the other single location indicates the present of another target body.

In another aspect, the invention is a detection system comprising

a stage for receiving a specimen field;

a detector positioned and configured to acquire images of locations within the specimen field at a set level and one or more additional amplifications of the set level;

a light source positioned and configured to expose the specimen field to light sufficient to excite a first fluorophore at a first excitation wavelength and sufficient to excite a second fluorophore at a second excitation wavelength;

a camera attached to the detector, the camera positioned and configured to (1) capture a first image at a location in the specimen field via an optical or electronic filter that substantially blocks photons at a second emission wavelength of the second fluorophore but is permissive for photons at a first emission wavelength of the first fluorophore, and (2) capture a second image at the location in the specimen field via an optical or electronic filter that substantially blocks photons at the first emission wavelength but is permissive for photons at the second emission wavelength; and

a computer that records the first image and second image and indexes the first image and second image to the corresponding location within the specimen field, the computer displaying, on demand by a user, the first image and second image for the corresponding location.

In other aspects, the system is any herein wherein the stage is movable about three perpendicular axes and addressable in at least two of the three axes; wherein the camera comprises a charge-coupled device for capturing the first and second images; wherein the camera comprises a plurality of optical filters; wherein the detector comprises a 1024×1024 pixel array image; wherein the detector comprises a 1600×1600 pixel array image; or wherein the detector comprises an A×B pixel array image, wherein A and B are each independently an integer between, 1000 and 1,000,000.

The invention also relates to a method for analyzing for biological agent cells in a specimen field of cells comprising:

i) treating the specimen field with a first fluorophore that identifies the biological agent cell;

ii) treating the specimen field with a second fluorophore that identifies the biological agent cell;

iii) exposing the specimen field with light suitable for causing the first fluorophore to emit photons,

iv) exposing the specimen field with light suitable for causing the second fluorophore to emit photons,

v) identifying cells in the specimen field that are emitting photons, which cells are biological agent cells.

In another aspect, the invention is any method herein: wherein the specimen field cell preparation comprises:

a. centrifuging a sample mixture;

b. resuspending the sample mixture;

c. plating the cells on an adhesive slide;

d. treating the slide with a fixative (paraformaldehyde);

e. treating the slide with a permeabilizing agent (Triton);

f. treating the slide with a pre-hybridization solution;

g. treating the slide with a hybridization solution having a fluorophore;

h. treating the slide with a fluorescent dye.

and that further comprising treating the specimen field with one or more additional fluorophore(s) that identifies the biological agent cell and exposing the specimen field with light suitable for causing the one or more additional fluorophore(s) to emit photons.

In other aspects, the invention relates to any method herein: wherein at least one fluorophore identifies DNA of a biological agent cell; wherein at least one fluorophore identifies a molecule that binds to the surface of the biological agent cell; wherein at least one fluorophore identifies DNA of a biological agent cell and at least one fluorophore identifies a molecule that binds to the surface biological agent cell; or wherein the biological agent is bacteria, Rickettsiae, viruses, fungi, or prions.

In another aspect, the invention is any method herein: wherein preparation of the specimen field comprises:

a. lysing the blood sample with ammonium chloride solution;

b. centrifuging the sample mixture;

c. separating the supernatant ammonium chloride solution and erythrocytes;

d. resuspending the resulting pellet of white cells in PBS;

e. centrifuging the sample mixture;

f. resuspending the resulting pellet of white cells in PBS;

g. making a dilution of the cell mixture of step f, tryphan blue, and PBS;

h. plating the cells on an adhesive slide;

i. adding cell culture media to the slide.

and that further wherein one fluorophore identifies cells that are not target cells. In other aspects the methods are those wherein the method is completed for a specimen field in less than 60 minutes; or wherein the method is completed for a specimen field in less than 10 minutes.

In other aspects, the invention is a method for screening a transplantation organ donor for the presence or absence of a target body comprising any method herein, wherein the specimen field is a sample (e.g., blood sample, tissue sample) taken from the organ donor. This is useful for identifying target bodies in the donor prior to transplantation, thus preventing spread of those bodies to the donee. The invention also relates to a method for assessing the efficacy of a drug candidate against a disease or disease symptom in a subject who was administered the drug candidate by screening for the presence or absence of a target body whose presence or absence is indicative of the disease or disease symptom comprising any method herein, wherein the specimen field is a sample taken from the subject. The invention also relates to a method for screening a blood sample for the presence or absence of a target body comprising any method herein, wherein the specimen field is a blood sample. This is useful for identifying contaminated blood samples, for example in blood banks, prior to distribution of those contaminated samples. It could also be used for screening potential donors prior to their donation. The invention is also a method for screening a fluid sample for the presence or absence of a target body comprising any method herein, wherein the specimen field is a fluid sample; and any method herein, wherein the target body is a cancer cell.

In another aspect, the invention is a method of screening for the presence of bacteria comprising any method herein: wherein at least one fluorophore comprises a DNA probe for bacteria; wherein the specimen field is taken from a surgical patient after surgery; wherein the specimen field is taken from a food sample; or any method herein further comprising:

j. exposing the slide to an aldehyde-based fixative;

k. rising the slide in phosphate-buffered saline (PBS);

l. adding human AB serum to the slide;

m. adding a primary antibody to the slide and incubating the slide;

n. rinsing the slide in PBS;

o. adding a secondary antibody to the slide and incubating the slide;

p. exposing the slide in an organic solvent;

q. rinsing the slide in PBS;

r. adding human AB serum to the slide;

s. adding a primary antibody to the slide and incubating the slide;

t. rinsing the slide in PBS;

u. adding a secondary antibody to the slide and incubating the slide;

v. rinsing the slide in PBS;

w. adding a cell dye to the slide and incubating the slide;

x. rinsing the slide with PBS;

y. exposing the slide to water;

z. mounting the slide;

or wherein the primary antibody in step s is keratin and the secondary antibody in step u is anti-rabbit rhodamine;

or any method herein further comprising:

j. exposing the slide in an organic solvent;

k. rinsing the slide in PBS;

l. adding a primary antibody to the slide and incubating the slide;

m. rinsing the slide in PBS;

n. adding a secondary antibody to the slide and incubating the slide;

o. rinsing the slide in PBS;

p. adding a cell dye to the slide and incubating the slide;

q. rinsing the slide with PBS;

r. exposing the slide to water;

s. mounting the slide;

or those: wherein the organic solvent is an alcohol or acetone; wherein the primary antibody is keratin; wherein the secondary antibody is anti-rabbit rhodamine; wherein the fluorophore detects bacteria; wherein the fluorophore is a nucleic acid probe; or wherein the nucleic acid probe is an oligonucleotide.

Other features or advantages of the present invention will be apparent from the following detailed description, and also from the claims.

### DETAILED DESCRIPTION

The invention relates to fluorescence-based methods and systems for detecting rare target bodies within a large number of candidate bodies. Because a wide variety of fluorophores are commercially available and have different peak emission wavelengths, the methods and systems can be adapted to detect many different target bodies within a single large population of candidate bodies. For example, fluorophores A, B, C, D, E, and F can be coupled to molecules that specifically bind to target bodies 1, 2, 3, 4, 5, and 6, respectively. One merely needs to capture and assess the emission wavelength, if any, of a candidate body and compare the emission wavelength with what would be expected from fluorophores A-F to determine whether the candidate body is a target body 1, 2, 3, 4, 5, or 6. In fact, far larger numbers of targets can be detected simultaneously in this manner. Additional details regarding the various reagents and procedures suitable for use in the invention are discussed below.

### Preparation of Specimens for Detection

In common clinical applications, a specimen will typically be a cell sample in body fluids, bone marrow, or a tissue sample, e.g., a blood cell sample, that can be screened for the presence of a rare cell having a particular phenotype (using, e.g., antibodies) or genotype (e.g., using oligonucleotide probes).

The cell specimen preparation methods herein result in enrichment for cell types desired for analysis. This can be accomplished by any suitable method for separating or isolating cells, including for example, gradient separation, or lysis and centrifugation.

For the automated detection of rare events in peripheral blood or bone marrow, it is important to utilize a preparation method with minimal cell loss during sample processing. Simple lysis of erythrocytes (e.g., using ammonium chloride solution) is preferred over Ficoll-based isolation methods to ensure maximal recovery of rare cells. Performing the lysis in the same tube containing the blood sample, then performing the separation (e.g., centrifuging, spinning down) in the same tube (i.e., involving no transfer of sample during the lysis and separation) also minimizes cell loss and minimizes cell representation variation in the sample (i.e., maintaining a consistent relative proportion of rare cells to other cells in the sample both before and after processing). The cell preparation/adhesion procedure described in the Example below yielded a homogeneous cell preparation.

In contrast, regular cytospin preparations can result in a loss of up to ⅔ of the cells. Information on cell number is unavailable for most studies using microscopic rare event detection because these studies fail to record the total number of cells actually being analyzed on the slides. Rather, these experiments merely relate the number of positive events to the total number of cells processed, assuming a complete recovery. This introduces a bias: not only was it found that cells are indeed inevitably lost during preparation, but the recovery can vary greatly between samples of a given type (see “Range” column in Table 1) as well as according to the type of sample analyzed. It was found that adhesive glass microscope slides from Marienfeld Laboratory Glassware (Paul Marienfeld GmbH & Co; www.superior.de) were excellent substrates for producing a cellular specimen field for subsequent fluorescence microscopy, because these slides were able to capture a homogenous cell monolayer (optimal cell density with minimal overlap). Once the media is introduced to the slide, treatment with any aldehyde-based fixative (e.g., paraformaldehyde, formalin, gluteraldehyde, cross-linking agent) fixes the cells. In certain cells types where the antigen is not at the cell surface, the cells can be permeablized, using a permeablizing agent (e.g., methanol, TRITON). If the antigen is a surface antigen, then the permeablization is not required. Exposure of the slides to an organic solvent (e.g., alcohols, ketones, methanol, ethanol, acetone) can be used to permeablize the cells, and certain solvents (e.g., methanol) can both fix and permeablize. Cell culture media can be any media that can cover free binding sites, or can have proteins, including for example RPMI or DMEM. Physiological buffer solutions are those that are compatible with cells and include for example, any isotonic solution, or PBS. Cell dyes are any dye suitable to stain a cell and include for example, DNA dyes, cytoplasmic dyes, mitochondrial dyes, DAPI, calcein and the like. With the proper specimen preparation, any unexpected cell type in a biological tissue or fluid can be detected using the invention. For example, the presence of smooth muscle cells in blood may indicate atherosclerosis. In another example, packaged blood in a blood bank can be screened for the existence of common pathogens transmitted by transfusion, such as human immunodeficiency virus, hepatitis B virus, or cytomegalovirus.

Whatever method is used to prepare the specimen field for analysis, it is important that the method does not destroy or significantly alter the target body to be detected. For example, if the target body is a prion, bacteria, virus, protozoan, or multicellular parasite, the isolation procedures may differ. Analysis of solid tissue (e.g., a solid tumor) may require disaggregating cells, e.g., by physical disruption instead of by trypsinization, since protease treatment can alter any cell surface molecule that is used to identify a target cell. Preparation of a virus specimen field may entail filtering out large particles of a certain size (e.g., cells) so that only sub-cellular particles are present in the specimen field. Alternatively, cells can be included in the specimen field if detection of virus-infected cells is desired. Various well known preparation procedures for particular biological samples are available to one skilled in the art of pathology and microscopy, and these procedures can be adapted to whatever target bodies are to be detected. Such procedures include cytospin using a Shandon Cytocentrifuge, Cytotek Monoprep from Sakura (Torrance, Calif.), and ThinPrep from Cysyc (Boxborough, Mass.).

When the sample to be analyzed is not a biological fluid such as blood, different devices can be used to collect samples from, e.g., air. In general, an air sampling device has a collection chamber containing liquid through or beside which air or gas is passed through, or containing a porous filter that traps particulates (e.g., target bodies) as air or gas passes through the filter. For collection chambers containing liquid, the collection liquid can be centrifuged or otherwise treated to separate particles from the liquid. The separated particles are then deposited onto a substrate for labeling or analysis. For collection chambers containing a filter (e.g., nitrocellulose), the filter can act as a substrate for subsequent labeling or analysis. Alternatively, particles can be washed from the filter, or the filter can be dissolved or otherwise removed from the particles. A filter collection chamber can also be adapted to collect particles from a liquid (e.g., water supply sample or cerebral spinal fluid) flowing through the filter. In addition, as discussed above, a liquid sample can be centrifuged to remove any particulate material present in the liquid. In instances when the test material remains in solution in the liquid sample and undesirable particulate matter is removed (e.g., by filtration), the mother liquor can be sampled (either in solution, or upon in vacuo drying of the sample solution) for analysis. A variety of samplers are known and available for use with the present invention. See SKC, Inc. (www.skc.com), which sells the SKC BioSampler® and other sampling devices.

It is contemplated that the invention encompasses detection of biological warfare agents or any agent that is harmful to humans, animals, or plants. In that light, the methods and systems of the invention can be used to detect agents harmful to humans, commercially valuable animals, or commercially valuable plants. Human bacteria and Rickettsiae agents include but are not limited to Coxiella burnetii, Bartonella Quintana (Rochalimea quintana, Rickettsia quintana), Rickettsia prowasecki, Rickettsia rickettsii, Bacillus anthraci, Brucella abortus, Brucella melitensis, Brucella suis, Chlamydia psittaci, Clostridium botulinum, Francisella tularensis, Burkholderia mallei (Pseudomonas mallei), Burkholderia pseudomallei (Pseudomonas pseudomallei), Salmonella typhi, Shigella dysenteriae, Vibrio cholerae, Yersinia pestis, Clostridium perfringens, Clostridium tetani, Enterohaemorrhagic Escherichia coli (serotype 0157 and other verotoxin producing serotypes), Legionella pneumophila, and Yersinia pseudotuberculosis. Human viral agents include but are not limited to Chikungunya virus, Congo-Crimean hemorrhagic fever virus, Dengue fever virus, Eastern equine encephalitis virus, Ebola virus, Hantaan virus, Junin virus, Lassa fever virus, Lymphocytic choriomeningitis virus, Machupo virus, Marburg virus, Monkey pox virus, Rift Valley fever virus, Tick-borne encephalitis virus, Variola virus, Venezuelan equine encephalitis virus, Western equine encephalitis virus, White pox, Yellow fever virus, Japanese encephalitis virus, Kyasanur Forest virus, Louping ill virus, Murray Valley encephalitis virus, Omsk hemorrhagic fever virus, Oropouche virus, Powassan virus, Rocio virus, and St. Louis encephalitis virus.

Animal bacteria and Rickettsiae agents include but are not limited to Mycoplasma mycoides and Bacillus anthracis. Animal viral agents include but are not limited to African swine fever virus, Avian influenza virus 2, Bluetongue virus, Foot and mouth disease virus, Goat pox virus, Herpes virus (Aujeszky's disease), Hog cholera virus (Swine fever virus), Lyssa virus, Newcastle disease virus, Peste des petits ruminants virus, Porcine enterovirus type 9 (swine vesicular disease virus), Rinderpest virus, Sheep pox virus, Teschen disease virus, and Vesicular stomatitis virus.

Plant bacteria and Rickettsiae agents including but not limited to Xanthomonas albilineans, Xanthomonas campestris pv. Citri, Xanthomonas campestris pv. Oryzae, and Xylella fastidiosa. Plant viral agents including but not limited to banana bunchy top virus.

Prions are correlated with diseases including but not limited to bovine spongiform encephalopathies, scrapi, and Creutzfeldt-Jakob disease.

In a particular example, a sample can be prepared as follows. Optimized preparation procedure for the immunocytochemical detection of microorganisms can be applied to environmental (air and water) and human (blood and other body fluids) samples. A BioSampler® from SKC, Inc. is used to collect an air sample. The BioSampler® is a vacuum-driven all-glass impinger device that passes air, via nozzles, tangential to the surface of the collection fluid rather than bubbling air through the fluid. This design minimizes particle bounce and reduces re-aerosolization. When operated at an air flow rate of 12.5 L/min with water or a liquid of similar viscosity as the collection fluid, the collection efficiency of the BioSampler is close to 100% for particles as little as 1 μm in diameter, still approximately 90% at 0.5 μm, and 80% at 0.3 μm. As such, the BioSampler® is an excellent device for the collection of airborne bacteria, fungi, pollen, and viruses, since most bacteria are between 1 and 10 μm in diameter and many viruses have a size in the lower end of this range (e.g. Ebola virus, 1000×80 nm).

Other air samplers can be used. For example, an alternative device is the Air-O-Cell sampling cassette (SKC, Inc.). In this device, the airborne particles are accelerated and made to collide with a tacky slide which is directly suitable for various staining procedures and microscopic examination. However, this collection method is inefficient for particles smaller than 2 or 3 μm.

The main parameters to be modified in environmental sampling are the time of sampling and the collection fluid composition. Various fluids can be tested and compared in direct inoculation tests with known amounts of organisms, for their capacity to support adhesion to the slides.

The analysis of human body fluids are exemplified by the analysis of blood samples, as described in Example 1 below.

### Fluorescent Staining

An advantage of the present invention is that the invention can be implemented using a large library of well known and publically available fluorescent molecules. Sources include, for example, Molecular Probes (Eugene, Oreg.), Jackson Immuno Research (West Grove, Pa.), Sigma (St. Louis, Mo.). These molecules are themselves capable of specifically binding to a portion of a target body (e.g., fluorescent DNA dyes), or can be coupled to antibodies or nucleic acids that specifically bind to portions of a target body. See, for example, Fluorescent and Luminescent Probes for Biological Activity, Ed. W T Mason, Academic Press, London, 1993 and Handbook of Fluorescent Probes and Research Chemicals by R P Haugland, Ed. M T Z Spence, Molecular Probes, 1996. In general, when antibodies are used in immunofluorescence, the fluorescent dye is chemically attached to a secondary antibody that binds to a primary antibody that is specific for an antigen on the target body or attached directly to a primary antibody. Primary antibodies are available for a wide variety of antigens. For example, if the target body is a prion, a prion-specific antibody can be used to detect prions in a patient's cerebral spinal fluid to diagnose Creutzfeldt-Jakob disease. Primary antibodies suitable for use include anti-GD2 and anti-GD-3 antibodies (Matreya Inc., Pleasant Gap, Pa.), anti-HER-2neu antibodies (Dako, Carpinteria, Calif.), anti-KSA/EpCAM antibodies (Dako) and anti-cytokeratin antibodies (Sigma, St. Louis, Mo.). Secondary antibodies suitable for use include those available from Molecular Probes (Eugene, Oreg.) and Jackson Immuno Research (West Grove, Pa.). Between antibody introduction steps in the slide preparation, PBS washes should be performed. If the antibody introduction, however, is a serum blocking reagent, that is, where the antibodies are introduced to block nonspecific binding sites in the sample, then a PBS wash is unnecessary or even undesirable.

The presence of so many different fluorophores, many of which have different peak excitation or emission wavelengths, enables multiplex detection of a large number (e.g., 24 or more) of target bodies within a specimen field. In this embodiment, each antibody can be specific for only one target body. In addition, multiplexing enables detection of nested groups of target bodies to provide greater detection accuracy (e.g., to minimize false positives). In the Example below, the DNA stain DAPI was used to identify target bodies that were nucleated cells, which can indicate total cell count in a sample and help confirm that a fluorescing marker is in fact associated with a cell, as opposed to a fragment or debris. Anti-cytokeratin antibodies were then used to identify candidate cancer cell targets within the target group of DAPI-positive cells. And finally, antibodies against surface cancer cell markers were used to identify and count the subgroup of true cancer cells that were DAPI-, cytokeratin, and cell surface antigen-positive. This nesting of fluorescence staining virtually eliminated false positive results. Other considerations are described below.

The first requirement for immunocytochemical assays is the generation of antibodies. When available commercially or otherwise, existing antibodies directed against surface or intracellular target antigens can be acquired. In other cases, the antibodies must be generated de novo. Irradiated (killed) samples of the organisms of interest can be obtained (e.g., pathogens from the CDC, USAMRIID, etc.) and provided to, e.g., A&G Pharmaceutical, Inc. (Baltimore, Md.) for the production of monoclonal antibodies (mAbs) to exposed epitopes. This company has developed a method for mAb production that provides for rapid development of hybridomas (<60 days) at a reasonable cost. If any of these organisms carry common surface epitope that would cause cross reaction, or if reliably “killed” organisms cannot be obtained, one or several antigens specific to the species can be obtained. In some situations, the target body to be detected is a class of targets and not an individual species within the class. Thus, an antibody that is class-specific rather than species-specific would be desirable. Antigens can be purified, expressed from their cloned genes, or mimicked by a chemically synthesized peptide. Antibodies can be directly conjugated with fluorescent molecules or used in combination with secondary fluorescently labeled antibodies. Directly labeled antibodies can be tested by FACS analysis for specificity against other phylogenetically related species.

The specificity of the detection of cancer cells in blood or bone marrow preparations is typically only as good as the marker and antibodies used in the procedure. The most widely used marker is cytokeratin, a cytoskeletal component of epithelial and carcinoma-derived cells. Although it has been validated as a valuable marker for breast, prostate, gastric, and colorectal cancer in a large number of clinical studies, cytokeratin is not a true tumor cell-specific marker and can stain epidermal cells, phagocytic cells that contain cytokeratin debris, or dye particles. In such cases, accurate microscopic confirmation of the malignant cytology of the immunostained cells is important. Another source of false-positive events is cross-reactive staining of the epithelial or cancer cell marker with blood or bone marrow cells, e.g. mucin-like epithelial membrane markers are able to cross-react with hematopoietic cells. Indeed, it was found that cytokeratin antibodies can label PBMC from healthy blood donors (Table 4 in Example 1). About 17% of the peripheral blood samples from normal blood donors exhibited cytokeratin positivity, albeit at a low level (mean was 1.18 CK+/10⁶ cells). It is not clear whether these CK+ cells in “normal” samples represent benign epithelial cells, cross-reacting hematopoietic cells, or cancer cells disseminated from an undiagnosed primary carcinoma.

To improve the specificity of cancer cell detection, a double-labeling protocol was developed for the simultaneous detection of cytokeratin and epithelial surface markers, Ep-CAM and GD2. This procedure dramatically reduced false positives, with only one doubly labeled cell among the 77 samples tested (CK/Ep-CAM and CK/GD2; Table 5 in the Example), suggesting that the few CK+ cells detected in normal samples were not of cancer origin. In addition to the mere detection of cancer cells in blood or bone marrow samples, efforts have been made to further characterize the phenotype of rare tumor cells, e.g. with respect to their aggressiveness, cell cycle stage, or growth behavior (Allgayer et al., J. Histochem. Cytochem. 45:203-212, 1997; Allgayer et al., Cancer Res. 57:1394-1399, 1997; Pantel et al., J. Natl. Cancer Inst. 85:1419-1424, 1993; and Riesenberg et al., Histochem. 99:61-66, 1993). Protocols for multiple marker analysis, combining cytokeratin labeling with growth factor receptors or proliferation-associated antigens to analyze breast cancer samples (Pantel et al., supra), or combining cytokeratin labeling with prostate specific antigen to analyze prostate carcinoma (Riesenberg et al, supra) have been developed. Also, in gastric cancer patients, cells that were doubly positive for cytokeratin and the urokinase plasminogen activator receptor correlated with high metastatic potential (Allgayer et al., Cancer Res. 57:1394-1399, 1997). A variety of possible additional (cancer-specific) markers have been described, e.g. glycoproteins (Franklin et al., Breast Cancer Res. Treat 41:1-13, 1996), gangliosides (Moss et al., N. Engl. J. Med. 324:219-226, 1991), cell adhesion molecules (Ross et al., Exp. Hematol. 23:1478-1483, 1995; and Ross et al., Bone Marrow Transplant. 15:929-933, 1995), and other molecules (Vrendenburgh et al., J. Hematother. 5:57-62, 1996). The sensitivity, quality, and specificity of the cancer cell detection method may improve as new markers become available.

Primary antibodies are available for a wide variety of antigens. For example, if the target body is a prion, a prion-specific antibody can be used to detect prions in a patient's cerebral spinal fluid to diagnose Creutzfeldt-Jakob disease.

Fluorescently labeled nucleic acids can be used as target body-specific probes instead of antibodies. Indeed, there are several reasons why detection using nucleic acid probes in an in situ hybridization (ISH) may be desirable: (1) Nucleic acid (NA) probes are easier, quicker, and cheaper to generate than antibodies (Abs); (2) NA probes can be grown at will and inexpensively (monoclonal Abs too, but not polyclonal); (3) NA probes are expected to be more consistent than Abs (especially polyclonal; can even choose probes with matching Tₘ, for multiple labeling (multiplex) experiments); (4) NA probe hybridization to its cognate RNA or DNA target can be better controlled than antibody interaction with its epitope (e.g., by hybridization temperature, ionic strength, etc.); (5) Multiple-label experiments are easier to implement with NA probes (simply incorporate a nucleotide conjugated to different labels, or incorporate biotin and then various streptavidin-label complexes; in immunofluorescence (IF), labeling of primary Ab may interfere with its binding, and when a second Ab is used for detection, IF requires the use of primary Abs raised in different species); and (6) Signals obtained with NA probes are expected to be more quantitative than with Abs, especially when directly labeled, yet can also be amplified if needed (biotin, etc.).

Using all the sequence information available on targeted bodies (e.g., biological warfare organisms), specific oligonucleotide probes to each of them can be designed. There is much less risk of stumbling onto a sequence shared with other organisms than is the case with cross-reacting epitopes, because each of the designed probes can be directly compared with the entire content of the bacterial/viral nucleic acids databases and designed to be unique to a particular target. Fairly short probes (e.g. 20-mers) can be used to maximize cell wall/capsid penetration and access to intracellular nucleic acid targets. The target sequence unique to a target body can be chosen to be on an abundantly expressed RNAs to maximize sensitivity, e.g., sequences in the ribosomal RNAs. For viruses, probes can be designed that are selective for the most abundantly expressed genes.

For single labeling experiments, the digoxigenin detection system (Zarda et al., J. Gen. Microbiol. 137:2823-2830, 1991) can be used. This system is commercially available as a kit from Boehringer Mannheim. In most instances, however, multiple labeling may be required, which is not possible with this system. Rather, the oligonucleotides can be synthesized in the presence of nucleotides conjugated to a fluorescent dye (e.g., one from Genset Corp.). If signal enhancement is required or sought, the oligonucleotides can be marked with a tag (e.g. biotin) during synthesis. In this case, each tagged probe would be reacted separately with one of several different streptavidin-label complexes, where the label is one of, for example, 24 fluorophores. These pre-reacted oligo probes complexes should be small enough to diffuse freely through bacterial membranes. If such is not the case, however, the cells can be permeabilized with lysozyme/EDTA.

As mentioned above, a wide variety of fluorescent molecules are known and available. It is estimated that over 50,000 dyes are available from Eastman Kodak, Polaroid, Fuji Film, and Molecular Probes (www.probes.com). Examples of molecules suitable for nucleated cell targets include DAPI, propidium iodide, acridine orange, and YOPRO.

### Detection System Components

The various components required for the detection systems are commercially available. The detector can be any means (e.g., instrument, combination of mirrors and/or lenses suitable, photomultiplier, or other detecting means) for measuring, recording, imaging, or detecting light, fluorescence or other energy transmission, including excitations, emissions, and the like. In general, the system includes a fluorescent microscope with a motorized stage (e.g., Nikon Microphot-FXA or Nikon Eclipse 1000, both from Nikon, Japan; stages from Ludl Electronic Products Ltd., Hawthorne, N.Y. or Axioplan 2 IE MOT from Zeiss, Germany), fluorescence filters (either included or made to order from Omega Optical, Brattleboro, Vt.), a camera (e.g., CCD 72 camera from DAGE-MIT, Inc., Michigan City, Ind.; AxioCam from Zeiss, Germany; or SpectraVideo camera from Pixelvision (www.pixelvision.com)), and a computer having a printer, monitor, storage medium, display, and software necessary for implementing the invention. Many of the listed components are available from vendors such as Nikon, Zeiss, Georgia Instruments (Roswell, Ga.), Vaytek (Fairfield, Iowa), Applied Imaging, Inc. (www.micrometastasis.org/metfs1.htm), and Chromavision Medical Systems, Inc. (www.chromavision.com).

Whatever components are used, the system should be capable of carrying out the following steps or variations and equivalents thereof:

1) counting the number of target bodies (e.g. cancer cells) per specimen field (e.g., a glass microscope slide), subdivided into categories of bodies containing the second or third fluorophore, or both;

2) saving (e.g., recording, imaging, storing on a data storage medium) an image of each target body;

3) storing the x,y coordinates for each target body; and

4) counting the total number of bodies on the slide.

The analysis is performed by scanning the specimen field. Scans can be performed at all magnifications provided by the microscope hardware. The user can choose to scan the specimen field using any filter set (single, dual, or triple). Scans can be run independently.

The algorithm for the detection and identification of target bodies is based on commercially available software for biological image analysis (e.g. Image Pro Plus from Media Cybernetics, www.mediacy.com; or KS 400 from Kontron, Germany). The inclusion criteria for the detection of target bodies can be for example:

a) fluorescence intensity threshold in the second and third fluorescent channels;

b) area and shape in the second and third fluorescent channels to distinguish true target bodies (e.g. intact cells) from false target bodies (e.g. dirt, debris); and

c) the signal(s) of the second and/or third fluorescent channels should always colocalize with the signal from the first fluorescent channel (e.g. DAPI signal).

Before each scan, the inclusion criteria for a target body are defined by the user. After the scan, a count for all target bodies that fulfill the inclusion criteria (see above) should be displayed and subdivided into target bodies that exhibit second, third, or both fluorescent labels. All target bodies that fulfill the inclusion criteria are imaged and stored as 3-color RGB-image (step 2 above). At the end of the scan, all images are displayed in form of a gallery of images with the option of zooming into each image. For all target bodies that fulfill the inclusion criteria (see above), the x,y-coordinates are stored and the user can recall each position and automatically move the stage to that position (step 3 above). This option allows the user to recheck every detected target body under high microscope magnification. It is also possible to recall the corresponding image that was taken at a specified position. During each scan, the total number of cells (based on the first fluorophore, e.g., DAPI signal) should be counted and displayed at the end of the scan (step 4 above).

### User Interphase with Detection System

1. Setup of the scan. At the beginning of the scan, the user is prompted to give the following information and to choose the parameters of the scan:

1. slide identification(s);

2. number of slides to be scanned;

3. magnification of the scan (choose objective); and

4. filter set(s) of the scan (choose between single, dual/triple filter, or alternate filters during the scan).

Based on the given information, an initial image is displayed and the camera is set up (adjust brightness and contrast). The user must define the inclusion criteria for the positive cells and choose:

1. intensity threshold;

2. lower and upper limit for the area; and

3. shape criteria.

2. Scan. After the initial setup, the scan starts automatically and analyzes the slide(s) according to the specifications.

3. Data output and storage. For each slide, the following information is displayed and saved:

1. number of target bodies;

2. image of each target body and corresponding coordinates on the stage; and

3. total number of target bodies on the slide.

The information 1-3 immediately above is stored in a folder named and defined by the user (identification of the slide).

4. Manual confirmation of positive cells. The user can manually select a stored image and recall the position were the image was taken. The stage automatically moves to that position and the field can be viewed through the eyepieces.

Speed is a fundamental parameter for evaluation of automated rare event analysis systems. The system described in Example 1 below takes about one hour to scan 1 million cells for positive events (e.g. CK positivity) and for the total cell count. Much faster systems may be employed, using a more sensitive charge-coupled device (CCD) camera and a faster computer. Such a system could bring down the processing time to a few minutes per million cells. This flow through rate is comparable to flow cytometry, yet retains the ability to observe each positive event at higher magnification or with different optics, for morphological confirmation if desired.

Without further elaboration, it is believed that one skilled in the art can, based on the above disclosure and the Examples below, utilize the present invention to its fullest extent. The following examples are to be construed as merely illustrative of how one skilled in the art can practice the invention, and are not limitative of the remainder of the disclosure in any way. All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLE 1

### Rare Event Imaging System for Cancer Cells

Materials & Methods

Collection of blood and bone marrow specimens. Five to ten milliliters of blood or bone marrow were drawn from control subjects or patients with a diagnosis of breast or small cell lung cancer and deposited in Vacutainer tubes containing EDTA as anticoagulant (Becton Dickinson, Franklin Lakes, N.Y.). All samples were obtained with informed consent from the subject or patient and were processed for microscopic analysis within 24 hours of collection.

Cell lines. The breast carcinoma cell line MCF-7 and the small cell lung cancer cell line SW2 were purchased from American Type Culture Collection (ATCC), Manassas, Va., and used to evaluate the staining protocol below and to determine the sensitivity of the Rare Event Imaging System. Cell lines were maintained in Dulbecco's modified Eagle's medium (MCF-7) or RPMI 1640 (SW2) containing 10% fetal calf serum, 100 U/ml penicillin, and 0.1 mg/ml streptomycin.

Sample preparation for microscopic analysis. Blood or bone marrow samples were mixed with 2 volumes of 0.17 M ammonium chloride, incubated at room temperature (RT) for 40 minutes, and centrifuged at 800×g for 10 minutes at RT. The cell pellet was then washed and resuspended in phosphate-buffered saline (PBS). The total number of living peripheral blood mononuclear cells (PBMC) or nucleated bone marrow cells was counted using Trypan blue dye exclusion. The cells were attached to adhesive slides (Paul Marienfeld GmbH & Co., KG, Bad Mergentheim, Germany) at 37° C. for 40 minutes, and the slides were then blocked with cell culture medium at 37° C. for 20 minutes. The total number of cells applied per slide was about 1.5×10⁶. The total adhesive area, divided into three separate circles, was about 530 mm².

For the single labeling of cytokeratin, cells were fixed in ice-cold methanol for 5 minutes, rinsed in PBS, and incubated with a rabbit anti-cytokeratin antiserum directed against class I and II cytokeratins (Biomedical Technologies, Stoughton, Mass.) at 37° C. for 1 hour. Subsequently, slides were washed in PBS, incubated with rhodamine-conjugated anti-rabbit antibody (Jackson Immuno Research, West Grove, Pa.) at 37° C. for 30 minutes, counterstained with 0.5 μg/ml 4′,6-diamidino-2-phenylindole (DAPI; Molecular Probes, Eugene, Oreg.) in PBS at RT for 10 minutes, and mounted in glycerol-gelatin (Sigma, St. Louis, Mo.). Processed slides were stored at RT and analyzed microscopically within a month.

For the double labeling of cytokeratin and the cell surface antigens Ep-CAM or GD2, the cells were fixed in 1% paraformaldehyde in PBS (pH 7.4) at RT for 5 minutes, washed in PBS, and blocked with 20% human AB serum (Nabi Diagnostics, Boca Raton, Fla.) in PBS at 37° C. for 20 minutes. Subsequently, primary antibodies directed against Ep-CAM (monoclonal mouse KS1/4 antibody) or GD2 (monoclonal mouse 1418 antibody) were applied at 37° C. for 1 hour. (Both antibodies were kindly provided by Dr. Kim-Ming Lo, Lexigen Pharmaceuticals, Lexington, Mass.) Antibodies directed against Ep-CAM are available from several vendors, e.g., monoclonal mouse anti-human epithelial specific antigen is available from Biomeda, Foster City, Calif.; monoclonal anti-human epithelial antigen (Ber-EP4) is available from Accurate Chemical & Scientific Corp., Westbury, N.Y.; and monoclonal HEA-FITC antibody is available from Miltenyi Biotec, Bergisch Gladbach, Germany. Antibodies directed against GD2 are available from Matreya, Inc., Pleasant Gap, Pa. Cells were then washed, fixed in ice-cold methanol for 5 minutes, blocked with 20% human AB serum, and incubated with anti-cytokeratin antiserum at 37° C. for 1 hour. Secondary antibodies (FITC-conjugated anti-mouse and rhodamine-conjugated anti-rabbit antibodies; Jackson Immuno Research) were mixed and applied at 37° C. for 30 minutes. Nuclei were counterstained with 0.5 μg/ml DAPI in PBS. Doubly labeled cells were mounted in Gel/Mount (Biomeda, Foster City, Calif.). Slides were stored at 40° C. and analyzed microscopically within a week.

Tumor cell dilutions for determination of sensitivity. To determine the sensitivity of the detection for cytokeratin-positive (CK+) cells, MCF-7 breast cancer cells were serially diluted in PBMC of a healthy blood donor. The dilutions tested were 1:10³, 1:10⁴, 1:10⁵, 1:2×10⁵, 1:5×10⁵, and 1:10⁶. Solutions were attached to adhesive slides and processed for cytokeratin labeling as described above. Up to 8 adhesive slides were prepared and scanned per dilution. Samples were analyzed for the number of tumor cells per slide and related to the total cell count.

Automated microscopic detection of tumor cells and total cell count. Slides were automatically scanned using an imaging system, such as for example, a Rare Event Imaging System, developed by Georgia Instruments, Inc. (Roswell, Ga.). The system employs proprietary image processing algorithms to detect rare fluorescent events and determine the total number of cells analyzed. It is comprised of an advanced computer-controlled microscope (Nikon Microphot-FXA, Nikon, Japan) with autofocus, motorized X, Y, and Z axis control, motorized filter selection, and electronic shuttering. Images were taken by an integrating, cooled CCD detector and processed in a 60 MHz Pentium imaging workstation.

In a first step, the slide was automatically scanned for the detection of positive events (e.g., CK+ cells) using the rhodamine filter set. The identification of positive events was based on fluorescence intensity and area. The (x,y) coordinates of each positive event were stored in computer memory, and the image was archived. In a second step, the slide was scanned for the total number of DAPI-labeled nuclei per slide, representing the total cell count. The total scanned area per slide was 448 mm² (84% of the adhesive area) to avoid edge effects. At the end of the two scans, the number of positive events and the total cell count were given, and a gallery of images containing all positive events was displayed. The user could review the images and recall any of the events for further examination, using the stored coordinates attached to each image. The field of interest could then be visualized using higher magnification and additional filter sets (e.g. fluorescein, or UV filter). Images of different fluorescent colors could be electronically overlaid for positive confirmation of the event and for phenotypic evaluation (multiple labeling). The total scanning time (two scans) for one slide was about 1 hour. The two scans could be run independently, offering the option of just screening for positive events and thus shortening the scanning time to 30 minutes per slide.

Results

Evaluation of the cell deposition procedure. One of the most critical steps during sample preparation is deposition of the cells onto slides. A qualitative microscopic comparison of cell preparations attached to poly-L-lysine/PBS-coated slides (0.1 %; Sigma, St. Louis), SectionLock Slides (Polysciences, Inc., Warrington, Pa.), and adhesive slides (Paul Marienfeld GmbH & Co., KG) revealed that the most homogeneous cell monolayers (optimal cell density with minimal overlap) was obtained with the slides from Paul Marienfeld. The slides contain a charged surface for the attachment of living cells. To further validate our deposition technique for different types of samples, the total number of cells as determined by the Rare Event Imaging System was compared with the number of cells originally deposited onto the slides. With optimization, any adhesive surface (e.g., coated with a positively charged substance such as poly-L-lysine) can be used. Table 1 shows a high cell recovery (89%) for peripheral blood of healthy blood donors, but a somewhat higher cell loss in samples from cancer patients (64, 58, and 73% recovery for PB, BM and SC samples, respectively; p<0.05 for PB and BM vs Normal PB, by t-test).

TABLE 1

| Sample | | | |
|---|---|---|---|
| type | Cell count/slide | Range (n) | Recovery |
| Normal | 1,120,237 ± 93,372 | 733,833-1,470,633 (8) | 89% |
| PB | | | |
| Cancer | 811,400 ± 89,039* | 223,393-1,473,777 (17) | 64% |
| PB | | | |
| Cancer | 731,945 ± 72,906* | 157,110-1,459,414 (25) | 58% |
| BM | | | |
| Cancer | 915,983 ± 95,806 | 76,745-1,631,660 (23) | 73% |
| SC | | | |

Peripheral blood (PB), bone marrow (BM), or peripheral blood stem cell (SC) samples from healthy subjects (Normal) or cancer patients were prepared as described in “Materials and Methods,” and 1.5×10⁶ cells were applied to each adhesive microscope slide. Cells were counted (based on DAPI labeling) on the number of slides indicated for each group (n), and results are expressed as mean±SEM. For the calculation of recovery, note that the area scanned on each slide is 84% of the total adhesive area (see “Materials & Methods”). Asterisks mean that p<0.05 vs Normal PB by t-test.

Sensitivity of the detection method. To explore the sensitivity of the Rare Event Imaging System, PBMC samples that had been spiked with breast cancer cells (MCF-7) were prepared and processed for cytokeratin labeling. The brightly stained epithelial MCF-7 cells could easily be distinguished from the mesenchymal background of the white blood cells. The sensitivity of detection of CK+ cells was tested with increasing tumor cell dilutions (MCF-7/PBMC) as described in “Materials & Methods.” Cancer cells in expected quantities could be detected up to the most diluted samples tested, 1 MCF-7 cell per 10⁶ PBMC (Table 2; expected and observed curves not statistically different, χ² test).

TABLE 2

| Cells added | Total number of | Total cell | Cells detected |
|---|---|---|---|
| per 10⁶ PBMC | cells detected | count | per 10⁶ PBMC |
| 1000 | 1789 | 1.94 × 10⁶ | 922 |
| 100 | 169 | 1.79 × 10⁶ | 95 |
| 10 | 27 | 2.35 × 10⁶ | 12 |
| 5 | 38 | 5.16 × 10⁶ | 7 |
| 2 | 11 | 3.94 × 10⁶ | 3 |
| 1 | 13 | 6.13 × 10⁶ | 2 |

Double-labeling of tumor cells. In order to increase the specificity of rare event detection and to further characterize the cancer cells identified, a staining protocol that allows the detection of intracellular cytokeratin and a cancer cell surface marker simultaneously was developed. The double-labeling procedure consists of two sequential steps: first fixing the cell surface and labeling for Ep-CAM or GD2, and second permeabilizing the cells and staining for intracellular cytokeratin. The double-labeling protocol was optimized in the cancer cell lines MCF-7 (breast cancer) and SW2 (small cell lung cancer). Fluorescence microscopy indicated that SW2 cells were efficiently labeled with anti-GD2 antibody and anti-cytokeratin antiserum. The sequential fixation preserved the antigenic sites of both proteins with regard to their cellular localization, as demonstrated in the optical sections taken with a confocal laser scanning microscope. The stained cells clearly showed cytokeratin in the cytoplasm (red) and GD2 at the cell surface (green). The expression levels of both proteins was quite heterogeneous within the cell population. A similar result was obtained when MCF-7 cells were doubly labeled with the anti-Ep-CAM antibody and the anti-cytokeratin antiserum. Control experiments in which one of the primary antibodies was omitted but both secondary antibodies were applied revealed no cross-reactivity between the two detection systems.

To further validate the staining protocol, PBMC that had been spiked with MCF-7 or SW-2 cells were labeled. The goal was to obtain a bright fluorescent signal of the cancer cells and a low background signal from the surrounding PBMC. The two most important factors for achieving this goal were found to be the sequential application of the primary antibodies and two blocking steps (20% human AB serum in PBS) prior to the incubation with the primary antibodies. Fluorescence microsocpy indicated that the doubly labeled MCF-7 cells could clearly be distinguished from the surrounding PBMC. At higher magnification, the intracellular cytokeratin labeling and the surface staining of Ep-CAM was confirmed. Similar results were obtained with PBMC spiked with SW-2 cells and doubly labeled for GD2 and cytokeratin.

The double-labeling protocol was also applied to peripheral blood and bone marrow samples from cancer patients. In an example of a GD2/cytokeratin-positive cell from peripheral blood of a patient with small cell lung cancer, fluorescence microscopy showed an Ep-CAM/cytokeratin-positive cell from bone marrow of a breast cancer patient. In this example, the cancer cell was not only bigger than the surrounding bone marrow cells but it also exhibits the distinct localization of the individual stains: cytokeratin (red) in the cytoplasm and Ep-CAM (green) concentrated towards the cell periphery at the cell membrane.

Detection of cytokeratin-positive and doubly positive cells in normal blood samples. To evaluate the specificity of the single- and double-staining protocols, blood samples from healthy donors were analyzed. The number of “positive” cells was compared among methods using the single cytokeratin or double cytokeratin/Ep-CAM or cytokeratin/GD2 labeling methods. Fluorescence microscopy indicated that 16-18% of the PB samples scored positive for cytokeratin using any of the protocols, with the number of CK+ cells ranging from 1 to 26 labeled cells per 10⁶ white blood cells. In contrast, when the samples were processed with the double-labeling protocol, positivity was almost completely eliminated from samples of healthy subjects (a single doubly positive cell was observed in a total of 77 PB samples).

Evaluation of spatial and temporal variations in sample collection. To assess a possible heterogeneity in the distribution of CK+ cells in different areas of the bone marrow, paired BM samples from the right and the left iliac crests of the same patient were obtained and analyzed. Out of 24 pairs, 21 showed concordant results (Fisher exact test) with regard to cytokeratin positivity (Table 3A). The occurrence of CK+ cells in peripheral blood samples temporal fluctuations was also tested. Two SC samples from each of 96 patient were taken at consecutive days but without therapeutic intervention. Paired samples showed a statistically significant concordance with regard to cytokeratin positivity (Table 3B).

TABLE 3A

BM 2

| | | + | − |
|---|---|---|---|
| BM 1 | + | 11 | 1 |
| | − | 2 | 10 |

TABLE 3B

SC 2

| | | + | − |
|---|---|---|---|
| SC 1 | + | 19 | 9 |
| | − | 8 | 60 |

Detection of cytokeratin-positive cells in cancer patient blood and bone marrow samples. To demonstrate the power of the Rare Event Imaging System, 355 peripheral blood, bone marrow, and stem cell samples were analyzed. These samples were obtained from breast cancer patients before autologous bone marrow transplantation but after high-dose chemotherapy. The samples were screened using the single cytokeratin labeling method. In an example of two CK+ cells from peripheral blood of a breast cancer patient, the positive cells showed clear cytoplasmic labeling whereas the surrounding blood cells were not stained. CK+ cells were found in 52% of the bone marrow, 34% of the peripheral blood, and 27% of the stem cell samples (Table 4).

TABLE 4

| | | CK+ samples | | CK+ samples | |
|---|---|---|---|---|---|
| | Total | (All) | | (≧9 CK+/10⁶ PBMC) | |
| | samples | Count | % | Count | % |
| BM samples | 63 | 33 | 52 | 25 | 40 |
| stages II/III | 20 | 7 | 35 | 5 | 25 |
| stage IV | 43 | 26 | 60 | 20 | 46 |
| PB samples | 59 | 20 | 34 | 14 | 24 |
| stages II/III | 13 | 2 | 15 | 2 | 15 |
| stage IV | 46 | 18 | 39 | 12 | 26 |
| SC samples | 233 | 64 | 27 | 29 | 12 |
| stages II/III | 49 | 11 | 22 | 4 | 8 |
| stage IV | 184 | 53 | 29 | 25 | 14 |

For Table 4, bone marrow (BM), peripheral blood (PB), and peripheral blood stem cell (SC) samples from a total of 156 patients were analyzed for cytokeratin-positive cells and total cell count. Note that there were multiple samples from some patients whereas for others, only one kind of sample could be analyzed. “CK+ samples (All)” refers to the number of samples with at least 1 CK+ cell. CK+ samples (≧9 CK+/10⁶ PBMC) refers to number of samples with 9 or more CK+ cells per 10⁶ PBMC (mean+2 SD of CK+ cells in Normal PB; Table 5). The highest numbers of CK+ cells per sample were 504/10⁶ for BM, 371/10⁶ for PB, and 1020/10⁶ for SC.

TABLE 5

| | | CK+labeled | | | Doubly labeled | |
|---|---|---|---|---|---|---|
| | | | | CK+/10⁶ | | DBL+/10⁶ |
| | Total | Positive | CK+/10⁶ | (CK+ | Positive | (DBL+ |
| Marker(s) | samples | samples | (all samples) | samples) | samples | sample) |
| CK | 57 | 10 (17%) | 1.18 ± 0.53 | 7.28 ± 2.59 | — | — |
| CK/Ep-CAM | 43 | 7 (16%) | 0.46 ± 0.21 | 2.85 ± 0.81 | 1 (2.3%) | 1.4 |
| CK/GD2 | 34 | 6 (18%) | 0.78 ± 0.44 | 4.41 ± 1.98 | 0 (0.0%) | 0 |

For Table 5, blood samples from healthy blood donors were labeled for cytokeratin alone, or doubly labeled for CK/Ep-CAM or CK/GD2 (see “Material and Methods”). Positive samples were those containing CK+ cells (in single-labeling) or doubly labeled cells (in double-labeling). Numbers of positive cells in each category are expressed per 10⁶ cells analyzed, and are given as mean±SEM (except for the single positive cell in one sample containing 7.14×10⁵ cells, in the CK/Ep-CAM group). DBL+ means doubly labeled

As seen in Table 4, the frequency of CK+ cells in the positive samples varied from 1/10⁶ to 1020/10⁶. However, many PB samples from normal subjects displayed a small number of CK+ cells, and these were found to be false positive cells, based on the double-labeling experiments (Table 5). Therefore, to declare definite positivity in PB samples from cancer patients, a cut-off point was set at the mean number of CK+ cells plus 2 times the standard deviation as observed in the control samples, i.e., 9/10⁶. Applying this threshold, a higher degree of cytokeratin positivity in bone marrow (40%) compared to peripheral blood (24%) or stem cell preparations (12%) was still found (Table 4). Furthermore, patients with stage IV disease were found to be cytokeratin-positive in a significantly higher percentage than patients with stages II/III disease, in all types of samples analyzed (Table 4).

In summary, an automated analysis system for the detection of cells of interest that occur at low frequencies (rare events) was developed using dual- or multiple-marker analysis. The preparation procedure for the microscopic analysis of blood or bone marrow samples was optimized for automation and included lysis of red blood cells, deposition of mononuclear cells onto adhesive sides, and immunofluorescent labeling of the sample. Slides were then examined at low magnification under a fluorescence microscope fitted with a motorized stage, and all the fluorescent events are imaged and catalogued in a computer database for later retrieval. For automated image analysis it is crucial to work with secondary antibodies that give a bright signal while maintaining a low background. We have recently tested dyes of the Alexa-series (Molecular Probes) that give very bright and stable fluorescence signals. Fluorescently labeled slides should be analyzed within one week. If longer storage is desired, a mounting medium that maintains stable fluorescent signals should be used. We found that the use of the ProLong Antifade Kit (Molecular Probes) gave excellent results after 3 month storage of the slides at 40° C.

### EXAMPLE 2

### Optimization of Rare Event Imaging System

Adaptation and optimization of basic procedures for sample preparation, cell attachment, and staining

The slides used (Paul Marienfeld GmbH & Co. KG, Bad Mergentheim, Germany) contain 3 adhesive circles of 150 mm² each, onto which the cells are seeded. The adhesion procedure developed for human cells is adapted to the processing of microorganisms. Selected parameters tested include the time of contact with the adhesive slide, the temperature, the pH, the composition of the buffer and its ionic strength. Separate tests are performed with a bacteria (e.g., E. coli, B. subtilis, V. cholerae) and viruses (e.g. reovirions) to verify for possible variability in their characteristics of adhesion to the slides. Detection is performed using DAPI or acridine orange (which labels RNA for RNA viruses). Since an even cell monolayer is essential for automation, testing with other cell deposition systems (e.g. cytospin using a Shandon Cytocentrifuge; Cytotek Monoprep from Sakura, Torrance, Calif.; and ThinPrep from Cysyc, Boxborough, Mass.) is used for comparison purposes.

Develop computer software for REIS automation

We have automated the whole sequence of steps for our original analyzer (Kraeft et al., Clin. Cancer Res. 6:434-442, 2000). As necessary, an appropriate camera driver is obtained for the particular system to be implemented. The writing of such drivers is well within routine skill in the art of computer programming. The REIS employs image processing algorithms to detect rare fluorescence events. Images are taken by the detector and processed in a PC-based imaging workstation. The software performs the detection of fluorescent signals (antigen-positive organisms) as well as total cell count (e.g., based on DAPI/acridine orange staining), automated signal positioning, image archiving, and image processing. Initially, this is be done with one or two fluorophores (e.g. AlexaFluor488 and AlexaFluor568). The multiplex detection system can be expanded to accommodate multiple dyes (e.g., up to 24 dyes), and the software superimposes each fluorescent signal observed with each dye, with the corresponding image obtained with, e.g., DAPI/acridine orange stain.

Improvement to the multiple labeling system

The methods herein are useful not only to monitor the presence of bacteria and viruses in air or water samples, but also to detect and identify pathogens, in particular those identified as BW agents. These are numerous, and although it would be possible to generate as many slides as there are agents to be tested for, this would be impractical. Rather, one aspect involves development of a multiplex system whereby various fluorophores are be used, whose excitation/emission spectra can be differentiated.

As discussed above, an estimated 50,000 fluorescent dyes are available. Thus it is possible to screen this collection for a set of at least 24 dyes that give the brightest fluorescent signals (for maximum sensitivity) whose excitation and emission spectra can be differentiated, and that can be conveniently conjugated to antibodies, via an isothiocyanate bridge. A set of dyes with similar fluorescence intensity would also be favorable.

A set of filters to match and discriminate at least the emission peaks of the dyes chosen, plus DAPI and acridine orange, are selected for use in the methods. The excitation wavelength is controlled either by a separate set of filters or by using a narrow-band prism for the incident light. The wheels carrying the fluorescence filters are modified to accommodate all the excitation and emission filter combinations required for the discrimination of the distinct fluorophores.

### EXAMPLE 3

### Development of Methods for Pathogen Detection by Immunocytochemistry

Generation of fluorescently labeled antibodies. The first requirement for immunocytochemical assays is the generation of good antibodies. When available commercially or otherwise, existing antibodies directed against surface antigens of BW agents are selected for use. In other cases, irradiated (killed) samples of the organisms of interest (from the CDC, USAMRIID, etc.) are selected for use and the production of monoclonal antibodies (mAbs) to exposed epitopes is performed; If any of these organisms carry common surface epitope that would cause cross reaction, or if reliably “killed” organisms cannot be obtained, one or several antigens specific to the species can be selected, and either purified, expressed from their cloned genes, or mimicked by a chemically synthesized peptide, and used as immunogens. All antibodies are either directly conjugated with fluorescent molecules or used in combination with secondary fluorescently labeled antibodies. Testing of directly labeled antibodies is performed by FACS analysis for specificity against other phylogenetically related species, especially those described herein.

Antibodies for a variety of bacteria, rickettsiae, viruses and fungi listed above or to other suitable model microorganisms can be used to develop a pathogen detection rare event imaging system (REIS). Each of six different antibodies is conjugated with 4 different dyes, for a total of 24 distinguishable fluorescently labeled antibodies. Ultimately, a multiplex detection system with 24 distinguishable fluorophores (conjugated to a set of 24 specific antibodies), would allow one to monitor and positively identify all the known or suspected BW agents directed to humans on only 2 slides (i.e. 2 sets of 24 antibodies), and nearly all the BW agents listed herein on only 3 slides.

Immunocytochemical detection of pathogens immobilized onto slides. Samples of attenuated strains or irradiated (killed) organisms for the 6 species to which antibodies are raised, (if pathogenic, from the CDC, USAMRIID, etc.), are applied in known numbers directly onto adhesive glass slides for analysis using the REIS.

In a first set of experiments, known numbers of organisms from each species individually, are examined to estimate the level of sensitivity of their cognate labeled antibodies, using the REIS; each should be able to detect single, individual organisms, and the enumeration should be quantitative. DAPI is used to stain DNA as before, or acridine orange for RNA viruses. All parameters of the procedure (temperature, buffer composition, antibody concentration, etc.) are optimized for each organism/antibody set, with a special attention to the minimization of the time of incubation. Initial conditions are essentially as described in Example 1.

In a second set of experiments, every pair of the organisms of interest, seeded on the slides in proportions of 10%-90% and 90%-10%, are examined to ascertain the absence of cross reactivity in the context of REIS analysis.

A third set of experiments test the multiplex set-up; using a mixture of 6 organisms (bacteria/rickettsiae, viruses, and mixture of the two), in the proportions of 5, 10, 15, 19, 23, and 28%, allowing for a verification of the detection efficiency using multiplex. Then, 24-strong multiplex experiments are performed using the 4 preparations of each of the 6 antibodies, with the organisms seeded in the same proportions as above.

Throughout these experiments, data is collected on detection efficiency. This can be performed on 75 organisms described herein (or any other BW agent). Such information is useful to determine sets of organisms that can be analyzed together efficiently, a point that may be especially important if, for example, the requirements for adhesion onto slides (see Example 2) or for the incubation with the antibodies varies between species.

Immunocytochemical detection of pathogens in environmental samples or human body fluids. As discussed above, optimized preparation procedures for the immunocytochemical detection of microorganisms are applied to environmental (air and water) and human (blood and other body fluids) samples. Whereas waterborne pathogens can be processed directly from the source, airborne bacteria and viruses require a special sampling procedure to immobilize them onto the slides. Several suitable air sampling devices exist on the market, including the BioSampler® from SKC. This is a vacuum-driven all-glass impinger device that uses air nozzles tangential to the surface of the collection fluid rather than bubbling air in the fluid, minimizing particle bounce and reducing re-aerosolization. When operated at an air flow rate of 12.5 L/min with water or a liquid of similar viscosity as the collection fluid, the collection efficiency of the BioSampler® is close to 100% for particles as little as 1 μm in diameter, and still approximately 90% at 0.5 μm and 80% at 0.3 μm. As such, the BioSampler® is an excellent device for the collection of airborne bacteria, fungi, pollen, and viruses: most bacteria are between 1 and 10 μm in diameter, and many viruses have a size in the lower end of this range (e.g. Ebola virus, 1000×80 nm). Other air samplers are also suitable. For example, an alternative from SKC which may be convenient for certain sample types is the Air-O-Cell sampling cassette, in which the airborne particles are accelerated and made to collide with a tacky slide which is directly suitable for various staining procedures and microscopic examination. However, this collection method is inefficient for particles smaller than 2 or 3 μm.

The main parameters to be modified in environmental sampling are the time of sampling, and the collection fluid composition. Various fluids can be tested and compared in direct inoculation tests with known amounts of organisms, for their capacity to support adhesion to the slides.

The analysis of human body fluids is exemplified by the analysis of blood samples. First, normal blood from donors it is spiked with a known amount of pathogen (bacteria and viruses). We will analyze the recovery of the microorganism, establish a detection threshold, and compare manual with automated analysis. Second positive blood cultures from microbiological laboratories (for example, the Dana-Farber Cancer Institute) are obtained and utilized in the detection method for bacteria described herein and compared with the clinical result (based on bacterial culture). Additionally, negative blood samples from patients can serve as controls.

Development of methods for pathogen detection by in situ hybridization. Even though immunofluorescence (IF) is currently the method of choice for Rare Event analysis, there are several reasons why detection using nucleic acid probes and an in situ hybridization (ISH) approach may be preferable. These are summarized in Table 7. Thus, such approaches offer useful alternatives to the use of antibodies in IF.

TABLE 7
Possible advantages of ISH over IF procedures

| | |
|---|---|
| 1. | Nucleic acid (NA) probes are a lot easier, quicker, and cheaper to |
| | generate than antibodies (Abs). |
| 2. | NA probes can be grown at will and inexpensively (monoclonal Abs |
| | too, but not polyclonal). |
| 3. | NA probes are expected to be more consistent than Abs (especially |
| | polyclonal; can even choose probes with matching Tm, for multiple |
| | labeling (multiplex) experiments). |
| 4. | NA probe hybridization to its cognate RNA target can be better |
| | controlled than antibody interaction with its epitope (hybridization |
| | temperature, ionic strength, etc.). |
| 5. | Multiple-label experiments are much simpler with NA probes (simply |
| | incorporate a nucleotide conjugated to different labels, or biotin and |
| | then various streptavidin-label complexes; in IF, labeling of primary |
| | Ab may interfere with its binding, and when a 2^{nd} Ab is used for |
| | detection, IF requires the use of primary Abs raised in different |
| | species). |
| 6. | Signals obtained with NA probes are expected to be more quantitative |
| | than with Abs, especially when directly labeled, yet can also be |
| | amplified if needed (biotin, etc.). |

Generation of nucleic acid probes. Using all the sequence information available on targeted organisms, we can design specific oligonucleotide probes to each of them. There is much less risk of stumbling onto a sequence shared with other organisms than was the case with cross-reacting epitopes (see Example 2) because each of the designed probes can be directly compared with the entire content of the bacterial/viral nucleic acids databases. Use of fairly short probes (e.g. 20-mers) can maximize cell wall/capsid penetration and access to intracellular nucleic acid targets, and abundantly expressed RNAs can be used to maximize sensitivity. In this instance, selection of sequences in the ribosomal RNAs to the cellular organisms of interest that are specific to each species is useful. For viruses, probes are designed to the most abundantly expressed gene.

For single labeling experiments, we can use the digoxigenin detection system, which is commercially available as a kit (Boehringer Mannheim). In most instances, however, multiple labeling may be required, which is not possible in this system. Rather, the oligonucleotides will be synthesized in the presence of nucleotides conjugated to the fluorescent dye (Genset Corp.). If signal enhancement is required or sought, we may mark the oligonucleotides with a tag (e.g. biotin) during synthesis. In this case, each tagged probe would be reacted separately with one of several different streptavidin-label complexes, where the label is one of the 24 fluorophores from above. These pre-reacted oligo probes complexes should still be small enough to diffuse freely through bacterial membranes. If such is not the case, one can permeabilize the cells with lysozyme/EDTA.

Detection of pathogens immobilized onto slides by in situ hybridization. These experiments will parallel those described in Example 2 above. The “first set of experiments” requires only single labels and is performed with the digoxigenin system. For all others, fluorescently labeled oligonucleotides or the biotinylated oligo +streptavidin-label complex detection method described above are used. Again, data collection on detection efficiency throughout these experiments can be evaluated to determine the optimal sets of BW agents (up to 24) that can be monitored together, as a multiplex assay on a single slide.

Detection of pathogens in environmental samples and human body fluids by in situ hybridization. These experiments parallel those described in Example 2. Environmental and human samples are analyzed in parallel, using immunocytochemistry and in situ hybridization.

### EXAMPLE 4

### Single Label Protocol

All antibodies are diluted in PBS containing 20% human AB serum.

1. Lyse blood: 11 ml isotonic NH₄Cl for 3 ml blood in a 15 ml conical tube. Leave at room temperature for 40 minutes.

2. Centrifuge at 1500 rpm for 5 minutes.

3. Remove supernatant of NH₄Cl and erythrocytes, leaving a pellet of white blood cells.

Resuspend in the pellet in 10 ml PBS.

4. Centrifuge at 1500 rpm for 5 minutes.

5. Remove supernatant; resuspend pellet in 1, 0.5, or 0.25 ml, depending on the size of the pellet.

6. Make a dilution of cells, trypan blue and PBS for cell counting on the haemocytometer. 1:100 DILUTION: 10 ul trypan blue, 10 ul cells, 980 ul PBS

7. Calculate and adjust to have 5×10ˆ 5 cells in 100ul per circle (on adhesion slide)

8. Plate cells on adhesive slides; allow 40 minutes for cell attachment at 37° C.

9. Add 60 ul of 50:50 media per circle to the slides; incubate at 37° C. for 20 minutes.

10. Put slides into 2% paraformaldehyde for 20 minutes, room temperature.

11. Rinse slides 2×3 minutes.

12. Put slides in −20° C. methanol for 5 minutes.

13. Rinse in PBS 2×3 minutes.

14. Add 60 ul of 20% human AB serum to each circle for 20 minutes at 37° C. DO NOT RINSE! Tap off.

15. Add 60 ul of primary antibody (e.g. anti-cytokeratin) to each circle. Incubate at 37° C. for 1 hour.

16. Rinse in PBS 2×3 minutes.

17. Add 60 ul of secondary antibody (e.g.anti-rabbit rhodamine). Incubate for 30 minutes, 37° C.

18. Rinse in PBS 2×3 minutes.

19. Add 60 ul of DAPI to each circle. Incubate at room temperature for 10 minutes.

20. Rinse in PBS 1×.

21. Place in dH2O.

22. Mount with Glycerol gelatin. Put 35 ul on each circle. Put on coverslip.

23. Slides may be stored at room temperature, covered with foil.

### EXAMPLE 5

### Double Label Protocol

All antibodies are diluted in PBS containing 20% human AB serum.

1. Lyse blood: 11 ml isotonic NH₄Cl for 3ml blood in a 15 ml conical tube. Leave at room temperature for 40 minutes.

2. Centrifuge at 1500 rpm for 5 minutes.

3. Remove supernatant of NH₄Cl and erythrocytes, leaving a pellet of white blood cells. Resuspend in the pellet in 10 ml PBS.

4. Centrifuge at 1500 rpm for 5 minutes.

5. Remove supernatant; resuspend pellet in 1, 0.5, or 0.25 mL, depending on the size of the pellet.

6. Make a dilution of cells, trypan blue and PBS for cell counting on the haemocytometer. 1:100 DILUTION: 10 ul trypan blue, 10 ul cells, 980 ul PBS

7. Calculate and adjust to have 5×10ˆ 5 cells in 100 ul per circle (on adhesion slide)

8. Plate cells on adhesive slides; allow 40 minutes for cell attachment at 37° C.

9. Add 60 ul of 50:50 media per circle to the slides; incubate at 37° C. for 20 minutes.

10. Place slides in 2% paraformaldehyde in PBS at room temperature for 20 minutes.

11. Rinse in PBS 2×3 minutes.

12. Add 60 ul of 20% human AB serum to each circle; leave on for 20 minutes, 37° C. Tap off. DO NOT RINSE!

13. Add 60 ul of primary antibody (e.g. anti-GD2, anti-GD3, anti-Her-2neu or anti-KSA/EpCAM) to each circle. Incubate at 37° C. for 1 hour.

14. Rinse in PBS 2×3 minutes.

15. Add 60 ul of secondary antibody (e.g. anti-mouse Alexa Fluor488). Incubate for 30 minutes, 37° C.

16. Rinse in PBS 2×3 minutes.

17. Put slides in −20° C. methanol for 5 minutes.

18. Rinse in PBS 2×3 minutes.

19. Add 60 uL of 20% human AB serum to each circle; leave on for 20 minutes, 37° C. Tap off. DO NOT RINSE!

20. Add 60 ul of primary antibody (anti-cytokeratin) to each circle. Incubate at 37° C. for 1 hour.

21. Rinse in PBS 2×3 minutes.

22. Add 60 ul of secondary antibody (e.g. anti-rabbit rhodamine). Incubate for 30 minutes, 37° C.

23. Rinse in PBS 2×3 minutes.

24. Add 60 ul of DAPI to each circle. Incubate at room temperature for 10 minutes.

25. Rinse in PBS 1×.

26. Rinse in dH2O. Let slides dry.

27. Mount slides with Pro-Long Anti-fade mounting media (Molecular Probes), coverslip and seal with clear nail polish (optional).

28. Store at 4° C., covered with aluminum foil.

### EXAMPLE 6

### Bacterial Sample Preparation

1. Grow bacteria to semi-log phase (OD @600 nm=0.5)

2. Spin 0.5 ml Bacteria Suspension (5 min @ 5K rpm)

3. Resuspend Bacteria in 1 ml of 10% PBS.

4. Add 50 ul of resuspended bacteria to well on slide (if use Clear-Cell or Ad-Cell slides do not require gelatin coating otherwise pretreat slides with gelatin to improve binding of bacteria to slide).

5. Dry bacterial suspension 37 Celsius for 30 min.

6. Rinse in PBS RT then place in 4% Paraformaldehyde soln for 25-30 min.

7. Rinse in PBS RT then drop 100 ul of Triton/well leave for 5 min

8. Rinse in PBS RT.

9. Add PreHyb soln 30 min @ 46 Celsius (Prehyb soln; 50 ng/ul BET42a in Hyb soln, Hyb soln see below)

10. *.Hyb for 120 min @46 Celsius. Hyb soln with Flourophore conc 5 ng/ul and BET42a conc 50 ng/ul

11. *Wash for 30 min @ 48 Celsius. Wash soln must be preheated to 48 Celsius. Run some Wash soln over slides before soaking in Wash soln for 30 min.

12. *Rinse slides RT PBS

13. *Apply DAPI RT for 5 min.

14. *Rinse DAPI off with RT PBS, AIR dry then Gel mount, cover slip and nail varnish the edges.

* steps from 10 on should be carried in DARK as fluorophore will photobleach.

Hyb Soln

| | |
|---|---|
| 40% Formaldehyde soln | |
| 5M NaCl | 720 ul |
| 1M Tris-HCl (pH 7) | 80 ul |
| 10% SDS | 4 ul |
| ddH2O | 1600 ul |
| Formaldehyde | 1600 ul |
| Add Fluorophore to get conc | 5 ng/ul |

Wash Soln

| | |
|---|---|
| 5M NaCl | 2.25 ml |
| 0.1% SDS | 0.1 ml |
| 1M Tris-HCl (pH 7.6) | 2.0 ml |
| ddH2O | 95.65 ml |

FITC, Alexa Fluor 488, Cy 5, Cy3, and Bet42a-blocking labeled oligonucleotides were examined. Experiments were performed to establish the specificity of the probes and signal intensity of the label. All of the above listed probe sequences reacted specifically with their targets but not with the other bacteria.

### EXAMPLE 7

### Patient Sampling

A male white patient diagnosed as having a malignant carcinoid tumor of the midgut had undergone treatment (don't know). Upon completing the standard treatment regimen, the patient was diagnosed as “cancer-free” based upon results of standard laboratory tests. The patient's blood was then subjected to the method essentially as described herein wherein the sample indicated a white blood cell count of 42×10⁶. A sample of 15×10⁶ of these white blood cells were plated and stained using anti-GD2 (14/18) and Alexa Fluor 488 antibodies as primary and secondary antibodies, respectively, followed by anti-cytokeratin and anti-rabbit rhodamine as primary and secondary antibodies, respectively, in the double label protocol as exemplified in Example 5. The patient's sample showed three positive cells detected by the cytokeratin antibodies. This result indicates that the patient was, in fact, not cancer-free. This detection resulted in the patient undergoing a further treatment regimen to attempt to more fully eradicate the cancerous cells in a timeframe such that the prognosis for an improved outcome is greater.

### Other Embodiments

It is to be understood that while the invention has been described in conjunction with the detailed description thereof, the foregoing description is intended to illustrate and not limit the scope of the invention, which is defined by the appended claims. Other aspects, advantages, and modifications are within the scope of this invention.

For example, although the procedures described in Example 1 are impressive, improvements of this “first generation” rare event imaging system can made. The limiting factor in some known cell processing rates is mechanical, as several million cells must be scanned at fairly high magnification (usually 10-20×). Indeed, system speed is a problem: at a processing rate of 1000 cells per minute, it would take about 3.5 days of non-stop data acquisition to scan 5×10⁶ cells. While the cell density on the slides might be increased somewhat, the size and sensitivity of the camera currently in use limit the magnification to 10× or 5× at best, which is insufficient and in all practicality precludes such slow systems from use in the detection of rare events when time is a factor. While considerably more rapid (up to 10⁷ cells in about 4 hours), “first generation” systems also need improvement for true usefulness in a clinical (or environmental monitoring) setting.

To address this shortcoming, a “second generation” REIS system can be employed, with a goal to shorten the microscope analyzing time from 4 hours to less than 10 minutes. Based on the technologies available in the rapidly growing electronic imaging and software industries, this goal is reasonable. The key is to use a very large field, extremely sensitive camera, which would allow the capture of large microscope fields without scanning the slide. The idea of using a high-gain digital camera to shorten the processing time of a “first generation” system came from the success of Hubble telescope. Far away stars can be captured by advanced digital (as opposed to analog) electronic cameras down to a single pixel. The size of the grade 1, high quantum efficiency, back-illuminated charge-coupled device (“CCD”) chip in some state-of-the art cameras is 24.5×24.5 mm, with a pixel array of 1024×1024. New CCD chips with a 1600×1600 pixel array are also available, which will allow one to survey even larger microscopic fields. Utilizing such technology, it is envisioned that the image of an entire slide could comprise a chip, and ultimately a cell imaged as a single pixel in a large specimen field (e.g., 1×10⁹ cells).

What is claimed is:

1. A method of detecting the presence or absence of a target body in a specimen, the method comprising
obtaining a specimen field exposed to or labeled with at least a first fluorophore and a second fluorophore, the first fluorophore emitting photons at a first wavelength and the second fluorophore emitting photons at a second wavelength;
exposing the specimen field to light sufficient to excite the first and second fluorophores;
scanning the specimen field at a low magnification for first sources of photons at the first wavelength and for second sources of photons at the second wavelength;
registering the location of each first source and each second source within the specimen field;
acquiring and recording a first image of the specimen field at each location, the first image generated via an optical or electronic filter that substantially blocks photons of the second wavelength but is permissive for photons of the first wavelength;
acquiring and recording a second image of the specimen field at each location at a high magnification, the second image generated via an optical or electronic filter that substantially blocks photons of the first wavelength but is permissive for photons of the second wavelength;
indexing each first image and each second image to the corresponding location within the specimen field; and
inspecting a first image and second image at a single location within the specimen field,
wherein the presence of a candidate body in the first and second images at the single location indicates the presence of a target body in the specimen.

2. The method of claim 1, wherein preparation of the specimen field comprises:
a. lysing the cell sample to give a sample mixture;
b. centrifuging the sample mixture;
c. separating the supernatant from the sample mixture;
d. resuspending the resulting pellet of cells in a physiological buffer solution;
e. plating the cells on an adhesive slide;
f. adding cell culture media to the slide.

3. The method of claim 2, wherein preparation of the specimen field further comprises:
after step d, making a dilution of the cell mixture, treating the dilution with a dye sensitive for dead cells, and performing a cell count to determine the sample cell density for the slide to be used.

4. The method of claim 2, wherein the target body is a cancer, epithelial, smooth muscle, dendritic, memory T-, memory B-, somatic, normal, aberrant, or stem cell.

5. The method of claim 2, wherein the system is capable of detecting at least one target cell in a specimen field of at least 1,000,000 cells.

6. The method of claim 2, wherein the recoding comprises at least a 1024×1024 pixel array image

7. The method of claim 2, wherein the field specimen comprises white blood cells as the majority of cell types.

8. A detection system comprising
a stage for receiving a specimen field;
a detector positioned and configured to acquire images of locations within the specimen field at a set level and one or more additional amplifications of the set level;
a light source positioned and configured to expose the specimen field to light sufficient to excite a first fluorophore at a first excitation wavelength and sufficient to excite a second fluorophore at a second excitation wavelength;
a camera attached to the detector, the camera positioned and configured to (1) capture a first image at a location in the specimen field via an optical or electronic filter that substantially blocks photons at a second emission wavelength of the second fluorophore but is permissive for photons at a first emission wavelength of the first fluorophore, and (2) capture a second image at the location in the specimen field via an optical or electronic filter that substantially blocks photons at the first emission wavelength but is permissive for photons at the second emission wavelength; and
a computer that records the first image and second image and indexes the first image and second image to the corresponding location within the specimen field, the computer displaying, on demand by a user, the first image and second image for the corresponding location.

9. A method for analyzing for biological agent cells in a specimen field of cells comprising:
i) treating the specimen field with a first fluorophore that identifies the biological agent cell;
ii) treating the specimen field with a second fluorophore that identifies the biological agent cell;
iii) exposing the specimen field with light suitable for causing the first fluorophore to emit photons,
iv) exposing the specimen field with light suitable for causing the second fluorophore to emit photons,
v) identifying cells in the specimen field that are emitting photons, which cells are biological agent cells.

10. The method of claim 9, wherein the specimen field cell preparation comprises:
i. centrifuging a sample mixture;
j. resuspending the sample mixture;
k. plating the cells on an adhesive slide;
l. treating the slide with paraformaldehyde;
m. treating the slide with Triton;
n. treating the slide with a pre-hybridization solution;
o. treating the slide with a hybridization solution having a fluorophore;
p. treating the slide with a fluorescent dye.

11. The method of claim 10, further comprising treating the specimen field with one or more additional fluorophore(s) that identifies the biological agent cell and exposing the specimen field with light suitable for causing the one or more additional fluorophore(s) to emit photons.

12. The method of claim 11, wherein at least one fluorophore identifies DNA of a biological agent cell.

13. The method of claim 10, wherein the biological agent is bacteria, Rickettsiae, viruses, fungi, or prions.

14. The method of claim 1, wherein preparation of the specimen field comprises:
a. lysing the blood sample with ammonium chloride solution;
b. centrifuging the sample mixture;
c. separating the supernatant ammonium chloride solution and erythrocytes;
d. resuspending the resulting pellet of white cells in PBS;
e. centrifuging the sample mixture;
f. resuspending the resulting pellet of white cells in PBS;
g. making a dilution of the cell mixture of step f, tryphan blue, and PBS;
h. plating the cells on an adhesive slide;
i. adding cell culture media to the slide.

15. A method for screening a transplantation organ donor for the presence or absence of a target body comprising the method of claim 2, wherein the specimen field is a sample taken from the organ donor.

16. A method for assessing the efficacy of a drug candidate against a disease or disease symptom in a subject who was administered the drug candidate by screening for the presence or absence of a target body whose presence or absence is indicative of the disease or disease symptom comprising the method of claim 2, wherein the specimen field is a sample taken from the subject.

17. A method for screening a blood sample for the presence or absence of a target body comprising the method of claim 2, wherein the specimen field is a blood sample.

18. A method for screening a fluid sample for the presence or absence of a target body comprising the method of claim 2, wherein the specimen field is a fluid sample.

19. The method of any of claims 15-18, wherein the target body is a cancer cell.

20. A method of screening for the presence of bacteria comprising the method of claim 2, wherein at least one fluorophore comprises a DNA probe for bacteria.

21. The method of claim 20, wherein the specimen field is taken from a surgical patient after surgery.

22. The method of claim 20, wherein the specimen field is taken from a food sample.

23. The method of claim 10, further comprising:
j. exposing the slide to an aldehyde-based fixative;
k. rising the slide in phosphate-buffered saline (PBS);
l. adding human AB serum to the slide;
m. adding a primary antibody to the slide and incubating the slide;
n. rinsing the slide in PBS;
o. adding a secondary antibody to the slide and incubating the slide;
p. exposing the slide in an organic solvent;
q. rinsing the slide in PBS;
r. adding human AB serum to the slide;
s. adding a primary antibody to the slide and incubating the slide;
t. rinsing the slide in PBS;
u. adding a secondary antibody to the slide and incubating the slide;
v. rinsing the slide in PBS;
w. adding a cell dye to the slide and incubating the slide;
x. rinsing the slide with PBS;
y. exposing the slide to water;
z. mounting the slide.

24. The method of claim 10, further comprising;
j. exposing the slide in an organic solvent;
k. rinsing the slide in PBS;
l. adding a primary antibody to the slide and incubating the slide;
m. rinsing the slide in PBS;
n. adding a secondary antibody to the slide and incubating the slide;
o. rinsing the slide in PBS;
p. adding a cell dye to the slide and incubating the slide;
q. rinsing the slide with PBS;
r. exposing the slide to water;
s. mounting the slide.

25. The method of any of claims 23 or 24, wherein the organic solvent is an alcohol or acetone.

26. The method of any of claims 23 or 24, wherein the primary antibody is keratin.

27. The method of any of claims 23 or 24, wherein the secondary antibody is anti-rabbit rhodamine.

28. The method of claim 23, wherein the primary antibody in step s is keratin and the secondary antibody in step u is anti-rabbit rhodamine.
